(19) United States
(12) Patent Application Publication
(10) Pub. No.: US 2014/0378895 A1
(43) Pub. Date: Dec. 25, 2014

(54) ENHANCED ELECTRIC BREAST PUMP

(71) Applicant: CLINICARE LTD., Raanana (IL)

(72) Inventor: Doron Barack, Raanana (IL)

(73) Assignee: CLINICARE LTD., Raanana (IL)

(21) Appl. No.: 14/046,974

(22) Filed: Oct. 6, 2013

Related U.S. Application Data
(60) Provisional application No. 61/837,201, filed on Jun. 20, 2013.
(60) Provisional application No. 61/862,023, filed on Aug. 4, 2013.

(51) Int. Cl. A61M 1/06

(52) U.S. Cl. CPC A61M 1/064
USPC 604/74; 700/90

(57) ABSTRACT

A breast pump includes a breast shield, a suction module for applying suction to the breast shield, and a controller. In one embodiment, the controller includes a wireless receiver for receiving pumping session descriptions according to which the suction module is operated. A corresponding non-volatile computer-readable storage medium has embodied thereon program code for defining pumping session descriptions and for transmitting the descriptions to the breast pump. In another embodiment, the controller includes a non-volatile memory for storing pumping mode descriptions, each of which includes a suction level, a cycle rate and an orientation, and a user interface for selecting one of the pumping mode descriptions for operating the suction module. In a third embodiment, the breast pump includes a port for reversibly coupling to an external memory for reading and/or writing pumping session descriptions and optionally for powering external devices.

This patent application claims priority from U.S. Provisional Patent Application No. 61/837,201, filed Jun. 20, 2013, and from U.S. Provisional Patent Application No. 61/862,023, filed Aug. 4, 2013.

### FIELD AND BACKGROUND OF THE INVENTION

The present invention relates to a breast pump and, more particularly, to a breast pump that receives values of operational parameters from either its own user interface or wirelessly from a remote device, and that associates the parameter values with a specific breast (right vs. left) of a user.

A breast pump is a mechanical device that extracts milk from the breasts of a lactating woman.

An electric breast pump is powered by a motor that supplies suction (partial vacuum) through tubing to a breast shield that is fitted over the nipple of the breast from which milk is being extracted.

In addition to the degree of partial vacuum, another important operating parameter of an electric breast pump is the cycle rate, which ideally is close to the 35-60 per minute rate of a suckling baby. In some high-end electrical breast pumps, such as the Medela Symphony™ breast pump, a higher cycle rate is used to stimulate lactation and a lower cycle rate is used for expression of milk.

Many high-end breast pumps are configured with manual user interfaces that users use to control the degree of partial vacuum and/or the cycle rate, and with memories for recording the last used degree of partial vacuum and/or cycle rate so that a user does not have to re-calibrate such a device all over again each time she uses the device.

### SUMMARY OF THE INVENTION

It would be useful for a breast pump to be configured to be operable wirelessly by a remote device such as a smart phone. A woman's preferred degree of partial vacuum and cycle rate could be recorded on the remote device and transferred from one such breast pump to another such breast pump. A log of such pumping sessions could be recorded and then transmitted to a lactation consultant.

Some women are more comfortable with different degrees of partial vacuum and different cycle rates on their right breasts vs. their left breasts. It would be useful for a breast pump (and, if the breast pump is configured for wireless operation, for the remote device) to associate an orientation (defined herein as meaning “right breast” vs. “left breast”) with a recorded pumping session, so that a user could retrieve and re-use the parameter values (degree of partial vacuum and cycle rate) appropriate for the breast that she wants to pump next.

Therefore, according to the present invention there is provided a breast pump comprising: (a) a breast shield; (b) a suction module for applying suction to said breast shield; and (c) a controller that operates the suction module in accordance with at least one description of a pumping session and that includes at least one of (i) a wireless receiver for receiving the at least one description, and (ii) a transmitter for transmitting the at least one description.

Furthermore, according to the present invention there is provided a non-volatile computer-readable storage medium having embodied thereon computer-readable code for remote operation of a breast pump, the computer-readable code comprising: (a) program code for defining at least one remote description of a pumping session; and (b) program code for transmitting said at least one remote description to the breast pump.

Furthermore, according to the present invention there is provided a breast pump comprising: (a) a breast shield; (b) a suction module for applying suction to said breast shield; and (c) a controller that includes: (i) a non-volatile memory for storing at least one description, of a respective pumping mode, that includes a respective suction level, a respective cycle rate and a respective orientation, and (ii) a user interface for selecting one of said at least one description, as stored in said memory, and for instructing said controller to operate said suction module in accordance with said one description.

Furthermore, according to the present invention there is provided a breast pump including: (a) a breast shield; (b) a suction module for applying suction to the breast shield; (c) a port for reversibly operationally coupling the breast pump to an external memory that is separate from the breast pump; and (d) a controller that operates the suction module in accordance with a description of a pumping session and that also is operative, when the external memory is operationally coupled to the breast pump at the port, to effect at least one operation selected from the group consisting of: (i) reading the description from the external memory, and (ii) writing the description to the external memory.

A first basic embodiment of a breast pump, according to the present invention, includes a breast shield, a suction module for applying suction to the breast shield, and a controller. The controller includes a wireless receiver for receiving one or more (usually two, one for stimulation and the other for expression) descriptions of a pumping session and/or a wireless transmitter for transmitting such (a) description(s) of a pumping session. The controller operates the suction module in accordance with the description(s).

Preferably, the controller also includes a memory for storing the description(s).

Preferably, the breast pump includes two or more breast shields, each such breast shield having a different respective tunnel size. The controller stores, along with each description of a pumping session, an identifier of the breast shield that was used during that pumping session.

Preferably, the breast pump also includes a user interface for entering the description(s).

Preferably, each description includes a respective suction level and a respective cycle rate.

Preferably, the transmitter transmits, along with each description of a pumping session, the duration of that pumping session.

Preferably, the breast pump includes two breast shields. Most preferably, the controller is operative to operate the suction module to apply suction to both breast shields in accordance with either the same description(s) or in accordance with different respective descriptions.

A corresponding basic non-volatile computer-readable storage medium has embodied thereon computer-readable code for remote operation of a breast pump.

The computer-readable code includes program code for defining one or more remote descriptions of pumping sessions and program code for transmitting the remote description(s) to the breast pump.

Preferably, each remote description includes a respective suction level and a respective cycle rate. Most preferably, one or more of the descriptions also include a creation date and/or an implementation date and/or an orientation (right breast vs. left breast).

Preferably, the computer-readable code also includes program code for storing each remote description in a non-volatile memory of the device that executes the computer-readable code.

Preferably, the computer-readable code also includes program code for receiving, from the breast pump, a respective duration of each of at least one pumping session that has been effected by the breast pump in accordance with (one of) the remote description(s). Most preferably, the computer-readable code also includes program code for storing the remote description of each such pumping session, along with the duration of the pumping session, in a non-volatile memory of the device that executes the computer-readable code.

Preferably, the computer-readable code also includes program code for receiving, from the breast pump, one or more local descriptions of pumping sessions. More preferably, each local description of a pumping session includes a respective duration, a respective suction level, a respective cycle rate and an identifier of the breast shield that was used during the pumping session. Most preferably, each local description also includes a creation date and/or an implementation date and/or an orientation (right breast vs. left breast) and/or a quantity of expressed milk.

Also more preferably, the computer-readable code also includes program code for storing each such local description in a non-volatile memory of the device that executes the computer-readable code.

Preferably, the computer-readable code also includes program code for displaying, at the device that executes the computer-readable code, one or more audio files and/or one or more video files.

A second basic embodiment of a breast pump, according to the present invention, includes a breast shield, a suction module for applying suction to the breast shield, and a controller. The controller includes a non-volatile memory and a user interface. The non-volatile memory is for storing one or more descriptions of pumping modes. Each pumping mode includes a respective suction level, a respective cycle rate and a respective orientation (right breast vs. left breast). The user interface is for selecting (one of) the description(s), as stored in the memory, and for instructing the controller to operate the suction module in accordance with the selected description.

Preferably, the user interface also is for providing the description(s) to be stored in the memory.

A third basic embodiment of a breast pump, according to the present invention, includes a breast shield, a suction module for applying suction to the breast shield, a port (such as a USB port) for reversibly operationally coupling the breast pump to a separate external memory, and a controller. The controller operates the suction module in accordance with a description of a pumping session. When the external memory is operationally coupled to the breast pump at the port, the controller is operative to read the description from the memory and/or to write the description to the memory.

Preferably, the breast pump also includes a user interface for entering the description.

Preferably, the controller also includes a wireless interface for transmitting and/or receiving the description.

Preferably, the port is a port, such as a USB port, that is operative to supply electrical power to an external device that is operationally coupled to the port.

### BRIEF DESCRIPTION OF THE DRAWINGS

Various embodiments are herein described, by way of example only, with reference to the accompanying drawings, wherein:

FIG. 1 is a diagram, partly in block form, of a breast pump of the present invention;

FIG. 2 is a high-level block diagram of a remote device for wireless operation of the breast pump of FIG. 1;

FIG. 3 shows a high-level block diagram of a server, as coupled to the Internet, for providing the app of the present invention to the remote device of FIG. 2.

### DESCRIPTION OF THE PREFERRED EMBODIMENTS

The principles and operation of a breast pump according to the present invention may be better understood with reference to the drawings and the accompanying description.

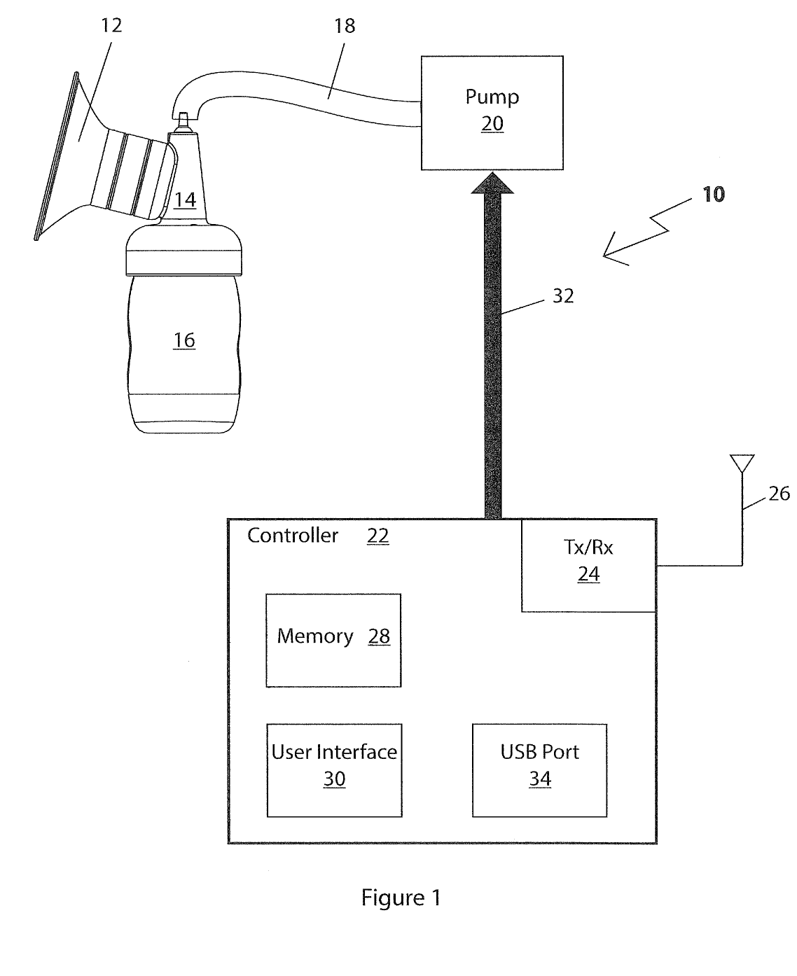

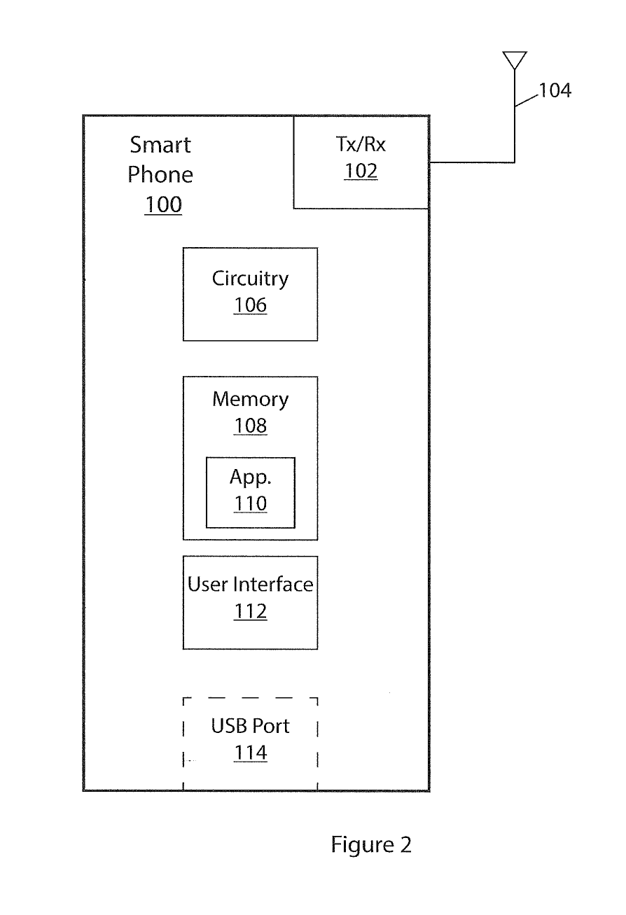

Referring now to the drawings, FIG. 1 is a diagram, partially in block form, of a breast pump 10 of the present invention, and FIG. 2 is a high-level block diagram of a remote device 100 (specifically, a smart phone) that is used for wireless operation of breast pump 10.

Breast pump 10 includes a breast shield 12 that is operationally coupled by a coupling portion 14 to a bottle 16 that screws into the base of coupling portion 14 and, via plastic tubing 18, to a vacuum pump 20. Coupling portion 14, plastic tubing 18 and pump 20 are components of a suction module for applying suction to breast shield 12. A decoupling element (not shown), such as a filter or a diaphragm, at the interface between tubing 18 and pump 20, blocks milk from being sucked into pump 20. Breast pump 10 also includes a controller 22 that operates pump 20 to apply partial vacuum, at a certain degree of partial vacuum (also called “suction level” herein) and at a certain cycle rate, to coupling portion 14, to extract milk from a breast to which breast shield 12 has been fitted. The control of pump 20 by controller 22 is represented symbolically in FIG. 1 by an arrow 32. The extracted milk is received into bottle 16.

Controller 22 includes a user interface 30 via which the user enters respective values of the two operational parameters (degree of partial vacuum, cycle rate) of the pumping sessions. These values are stored in a non-volatile memory 28. A “pumping session” may be either a simple pumping session for either the right breast or the left breast, with a single respective value of the degree of partial vacuum and a single respective value of the cycle rate, or a compound pumping session, that consists of two or more consecutive simple pumping sessions. For example, a user could use, in a simple pumping session, the same values of degree of partial vacuum and cycle rate for both stimulation and expression, or could use, in a compound pumping session that includes two simple pumping sessions, one pair of parameter values for stimulation and another pair of parameter values for expression. The “pumping sessions” that are recited in the appended claims are simple pumping sessions.

The respective parameter values of each simple pumping session, whether an isolated simple pumping session or a member of a compound pumping session, are stored in memory 28, along with the duration of the simple pumping session. User interface 30 also may be used to retrieve, from memory 28, the operational parameters of a previous simple or compound pumping session for use in a new pumping session. User interface 30 is of conventional construction, and includes e.g. a touch screen for input and output, and, optionally, one or more buttons, such as an on/off button, for user input.

Controller 22 is also configured to allow a user to enter, via user interface 30, an identification of which breast (right or left) she is extracting milk from in a particular pumping session. This identification is stored in memory 28 as part of the description of the pumping session, to give the user the option of later retrieving and using different parameter values for pumping her right breast than for pumping her left breast.

Controller 22 is also configured to allow a user to enter, after a simple or compound pumping session, the quantity of milk expressed in that session, as determined, e.g., with the help of graduations on the sides of bottle 16.

Controller 22 also includes a USB port 34 for operationally interfacing with an external nonvolatile memory such as a flash disk. Controller 22 is configured to both write descriptions of pumping sessions (whose parameters have been provided e.g. via user interface 30) to the external memory and to read descriptions of pumping sessions from the external memory. This feature of the present invention enables a user who has determined the parameter values she likes best to share those parameter values among several breast pumps 10. The external memory also serves as a backup to internal memory 28. In addition, because a USB port can provide electrical power at 5 V and up to 900 mA, USB port 34 may be used for charging external devices generally.

Controller 22 also includes a transceiver 24 (i.e. a combined transmitter and receiver) and an antenna 26 for wirelessly receiving values of pumping session operational parameters from a remote device such as a suitably configured smart phone, and for wirelessly transmitting descriptions of the consequent pumping sessions, or of pumping sessions whose parameters have been entered at user interface 30, to the remote device.

One of the uses of such a remote device is as an alternative to the external memory that couples to USB port 34. Other uses of the remote device are described below.

FIG. 2 is a high-level block diagram of such a remote device, specifically, a smart phone 100. Smart phone 100 is a conventional smart phone that includes a conventional transceiver 102 and antenna 104 for communicating wirelessly with other suitably equipped devices (including, inter alia, breast pump 10), conventional operational circuitry 106, a conventional user interface 112 (including e.g. a touch screen, a speaker and a microphone), and a conventional non-volatile memory 108 for storing user applications (“apps”), as is known in the art. The aspect of smart phone 100 that is germane to the present invention is that one of those apps is an app 110 for remotely controlling breast pump 10. App 110 gives smart phone 100 all the functionality of user interface 30 of breast pump 10. App 110 enables a user of smart phone 100 to enter values of operational parameters for simple or compound pumping sessions; to save, in memory 108 or in a similar memory of smart phone 100 records of pumping sessions (including respective parameter values, respective durations, and, optionally, respective quantities of expressed milk and/or respective breast identifications, as well as creation dates of the parameter values and implementation dates of the pumping sessions), thereby creating a log of pumping sessions; and to retrieve such records so that the parameter values recorded in the records may be re-used or to transmit the records to a lactation consultant. The records may be records of pumping sessions whose parameter values were defined “remotely” at smart phone 100 or whose parameter values were defined “locally” at breast pump 10. Hence, in some of the appended claims, a pumping session description that includes operational parameters as entered at user interface 30 is called a “local” description and a pumping session description that includes operational parameters as entered at user interface 112 under the control of app 110 is called a “remote” description

Note that an external memory interfaced to USB port 34 also could be used to maintain a log of pumping sessions.

App 110 preferably also enables smart phone 100 to be used to entertain the user while she is expressing milk, for example by displaying audio-visual content (stored in audio and/or video files in smart phone 100 as part of app 110 or as part of a different app, or received via transceiver 102) at user interface 112 or by playing music (also stored in smart phone 100 as part of app 110 or as part of a different app, or received via transceiver 102) at user interface 112.

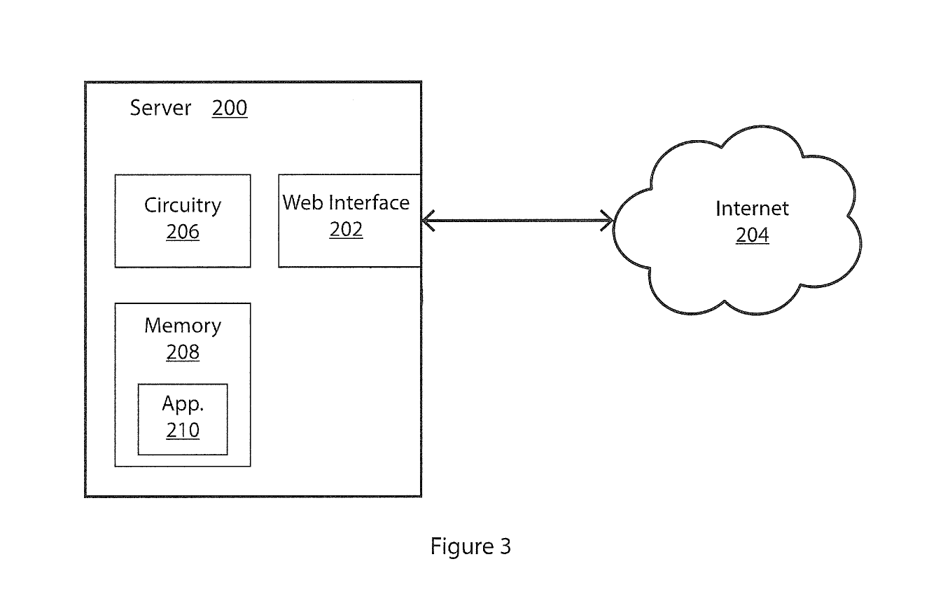

The user of breast pump 10 and smart phone 100 purchases app 110 from a vendor of app 110 in the conventional manner. FIG. 3 shows a server 200, coupled to the Internet 204 via a conventional Web interface 202, that the vendor may use for selling app 210. Server 200 includes conventional operational circuitry 206 and a non-volatile memory 208 wherein is stored app code 210 of the present invention. A user of breast pump 210 and smart phone 100 who wishes to purchase app 110 and install app 110 in smart phone 100 communicates with server 200 in the conventional manner. In response to a successful purchase request, server 200 sends the code of app 110 to the purchaser in the conventional manner. The app of the present invention is designated by the reference numeral “210” in FIG. 3 instead of by the reference numeral “110” because the code actually stored in server 200 may not be identical to the code that is sent to the purchaser, e.g. for reasons of security.

Memories 108 and 208 are examples of non-volatile storage media that have embedded thereon computer-readable code for remote operation of a breast pump as described herein.

Optionally, smart phone 100 is provided with a USB port 114, to give the user the option of transferring pumping session descriptions between smart phone 100 and breast pump 10 using an external memory such as a flash disk, instead of via transceivers 24 and 102.

One of the structural components of breast shield 12 is its tunnel. The partial vacuum created by pump 20 draws the user's nipple into the tunnel. A woman's nipples may change in size during the course of nursing, leading her to switch from one breast shield 12 with one tunnel size to another breast shield 12 with another tunnel size. Therefore, preferably, breast pump 10 is provided with several breast shields 10, each with its own respective tunnel size, and each pumping session record, as stored on breast pump 10 and/or as stored on smart phone 100, includes an identifier of the breast pump 12 that was used in that pumping session.

FIG. 1 shows breast pump 10 with a single breast shield 12 operationally coupled to pump 20. Optionally, breast pump 10 is configured so that two breast shields 12 are operationally coupled simultaneously to pump 20. Pump 20 then may be used by a user to extract milk from both of her breasts simultaneously. The same values of the operating parameters (suction level and cycle rate) may be used for both breasts. Alternatively, different respective values of the operating parameters may be used for the two breasts.

To the extent that the appended claims have been drafted without multiple dependencies, this has been done only to accommodate formal requirements in jurisdictions which do not allow such multiple dependencies. It should be noted that all possible combinations of features which would be implied by rendering the claims multiply dependent are explicitly envisaged and should be considered part of the invention.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A breast pump comprising:
(a) a breast shield;
(b) a suction module for applying suction to said breast shield; and
(c) a controller that operates said suction module in accordance with at least one description of a pumping session and that includes at least one of:
(i) a wireless receiver for receiving said at least one description, and
(ii) a transmitter for transmitting said at least one description.

2. The breast pump of claim 1, wherein said controller also includes a memory for storing said at least one description.

3. The breast pump of claim 1, comprising a plurality of said breast shields, each said breast shield having a different respective tunnel size, and wherein said controller stores, along with each said at least one description of a pumping session, an identifier of said breast shield that was used during said pumping session.

4. The breast pump of claim. 1, further comprising:
(d) a user interface for entering said at least one description.

5. The breast pump of claim 1, wherein each said description includes a respective suction level and a respective cycle rate.

6. The breast pump of claim 1, wherein said transmitter is for transmitting said at least one description along with a duration of said pumping session.

7. The breast pump of claim 1, comprising two said breast shields.

8. The breast pump of claim 7, wherein said controller is operative to operate said suction module to apply said suction to both said breast shields in accordance with a common said at least one description.

9. The breast pump of claim 7, wherein said controller is operative to operate said suction module to apply said suction to each said breast shield in accordance with a respective said at least one description.

10. A non-volatile computer-readable storage medium having embodied thereon computer-readable code for remote operation of a breast pump, the computer-readable code comprising:
(a) program code for defining at least one remote description of a pumping session; and
(b) program code for transmitting said at least one remote description to the breast pump.

11. The non-volatile computer-readable storage medium of claim 10, wherein each said remote description includes a respective suction level and a respective cycle rate.

12. The non-volatile computer-readable storage medium of claim 11, wherein at least one said remote description also includes at least one datum selected from the group consisting of a creation date, an implementation date and an orientation.

13. The non-volatile computer-readable storage medium of claim 10, wherein the computer-readable code further comprises:
(c) program code for storing each said remote description on a non-volatile memory of a device that executes the computer-readable code.

14. The non-volatile computer-readable storage medium of claim 10, wherein the computer-readable code further comprises:
(c) program code for receiving, from the breast pump, a respective duration of each of at least one said pumping session that has been effected by the breast pump in accordance with one of said at least one remote description.

15. The non-volatile computer-readable storage medium of claim 14, wherein the computer-readable code further comprises:
(c) program code for storing said remote description of each said at least one pumping session, along with said respective duration of each said at least one pumping session, in a non-volatile memory of a device that executes the computer-readable code.

16. The non-volatile computer-readable storage medium of claim 10, wherein the computer-readable code further comprises:
(c) program code for receiving, from the breast pump, at least one local description of a pumping session.

17. The non-volatile computer-readable storage medium of claim 16, wherein each said local description of a pumping session includes a respective duration, a respective suction level, a respective cycle rate and an identifier of a breast shield that was used during said pumping session.

18. The non-volatile computer-readable storage medium of claim 17, wherein at least one said local description also includes a datum selected from the group consisting of a creation date, an implementation date, an orientation and a quantity of expressed milk.

19. The non-volatile computer-readable storage medium of claim 16, wherein the computer-readable code further comprises:
(c) program code for storing each said local description in a non-volatile memory of a device that executes the computer-readable code.

20. The non-volatile computer-readable storage medium of claim 10, wherein the computer-readable code further comprises:
(c) program code for displaying, at a device that executes the computer-readable code, at least one file selected from the group consisting of an audio file and a video file.

21. A breast pump comprising:
(a) a breast shield;
(b) a suction module for applying suction to said breast shield; and
(c) a controller that includes:
(i) a non-volatile memory for storing at least one description, of a respective pumping mode, that includes a respective suction level, a respective cycle rate and a respective orientation, and
(ii) a user interface for selecting one of said at least one description, as stored in said memory, and for instructing said controller to operate said suction module in accordance with said one description.

22. The breast pump of claim 21, wherein said user interface also is for providing said at least one description to be stored in said memory.

23. A breast pump comprising:
(a) a breast shield;
(b) a suction module for applying suction to said breast shield;
(c) a port for reversibly operationally coupling the breast pump to an external memory that is separate from the breast pump; and
(d) a controller that operates the suction module in accordance with a description of a pumping session and that also is operative, when said external memory is operationally coupled to the breast pump at said port, to effect at least one operation selected from the group consisting of:
(i) reading said description from said external memory, and
(ii) writing said description to said external memory.

24. The breast pump of claim 23, further comprising:
(e) a user interface for entering said description.

25. The breast pump of claim 23, wherein said controller also includes a wireless interface for effecting at least one operation selected from the group consisting of:
(i) transmitting said description, and
(ii) receiving said description.

26. The breast pump of claim 23, wherein said port also is operative to supply electrical power to an external device that is operationally coupled to said port.
